(19) United States
(12) Patent Application Publication
(10) Pub. No.: US 2007/0155002 A1
(43) Pub. Date: Jul. 5, 2007

(54) Amino acid sequence from active principles in musk and their acetic salts, preparation and use thereof

(76) Inventor: Wenyan Wu, Guangdong (CN)

Correspondence Address: TRASK BRITT, P.O. BOX 2550, SALT LAKE CITY, UT, 84110, US

(21) Appl. No.: 10/585,276

(22) PCT Filed: Nov. 26, 2004
(86) PCT No.: PCT/CN04/01362

§ 371 Date: Nov. 30, 2006

(30) Foreign Application Priority Data
Jan. 5, 2004 (CN) 200420000851.2

(51) Int. Cl. C12N 9/00

(52) U.S. Cl. 435/183

(57) ABSTRACT

The present invention provides amino acid sequences obtained from the active principles in Chinese traditional medicine musk, such as SDSECPLLCEVWILK, SDSECPLLPRQGTGSLH, IDCECPLLEAKCPSFPLWPQGREEERQ, SDSECPLLLNGTNTSSRFESINCVFLSTEEGC and their acetic salts. The preparation of the amino acid sequences and their use as anti-inflammatory medicines or immunity inhibitors are also disclosed by this invention.

### TECHNICAL FIELD

The present invention relates to amino acid sequences from the active principles in traditional Chinese medicine musk and their acetic salts.

### BACKGROUND OF INVENTION

Identification of the active principles in traditional Chinese medicine (TCM) is a prerequisite for modernization of TCM. We have identified the main components and active principles in hundreds of commonly used medicinal herbs or natural products and the medical effects of many of the active principles. However, the research in the active principles in TCM, in general, is far from being complete. For example, many components identified have not been correlated with clinical effects. Fundamental theories and techniques in the research of the active principles of TCM need to be improved, and new concepts and novel technologies need to be introduced. Nowadays, with the development of gene technology, proteins and peptides in TCM should be the focus for the research of active principles in TCM.

For a long time, the commonly used methods to identify the active principles, such as flavones, sterol or glycoside, in TCM have been limited to chemical or physical analysis. Researchers have not paid much attention to peptides and proteins. Proteins and peptides are important components in various biological functions. Research on bioactive peptides and their related genes may lead to discovery of novel therapeutics or medicinal precursors.

Research has shown that musk contains about 25% protein, but the molecular structures of musk's polypeptides have not been reported yet. At present, some chemical compositions, such as muscone, muscopyridine and C19 steroid, can be synthesized.

During the Sixth and Seventh Five-year Plan periods, our government organized a series of key scientific researches to obtain the composition spectrum of natural musk and reconstruct artificial musk. Our researchers have obtained from natural musk the musk's polypeptide, which polypeptide comprises 15 amino acids, and have demonstrated that the inhibitive effect of musk's polypeptides to inflammation in rat's ear caused by croton oil is 36 times (based on mg/kg) or 500 times (based on molarity) stronger than that of hydrocortisone. It has been proven that musk polypeptides are indispensable ingredients for musk to exert therapeutic effects. However, when TCM musk was submitted for approval as a new medicine, it was replaced by some artificial product (e.g., fanghuosu) because of the failure to synthesize musk's polypeptides. This is one of the reasons that people still prefer natural musk.

Since musk's polypeptides have stronger effects than glucocorticoids, it can be speculated that musk's polypeptides may have important therapeutic effects for immune-mediated tissue injuries and diseases, such as allergic asthma, glomerulonephritis, systemic lupus erythematosus, rheumatoid arthritis, pustular vasculitis, myasthenia gravis, diabetes, etc. To date, these injuries and diseases are still treated with glucocorticoids, which lead to toxic side effects caused by steroid hormone. Therefore, it is still very difficult to treat these diseases. In general, musk's polypeptides have great values in clinical applications and a huge market prospect.

### OBJECTIVE

The objective of the invention is to provide polypeptide sequences obtained from the active principles in TCM musk and their preparation method.

### TECHNIQUES USED IN THE INVENTION

In order to utilize the active principles from musk as medicines, the invention provides amino acid sequences obtained from the active principles in TCM musk such as:

SDSECPLLCEVWILK
SDSECPLLPRQGTGSLH
IDCECPLLEAKCPSFPLWPQGREERQ
SDSECPLLLNGTNTSSRFESINCVFLSTEEGC

and their respective acetic salts such as:

(SDSECPLLCEVWILK)Ac
(SDSECPLLPRQGTGSLH)Ac
(IDCECPLLEAKCPSFPLWPQGREERQ)Ac
(SDSECPLLLNGTNTSSRFESINCVFLSTEEGC)Ac

The invention also provides a method to obtain amino acid sequences from the active principles in TCM, characterized in: separating and purifying the protein and polypeptide extracted from the functionally active parts that contain active principles of TCM and obtaining active polypeptides or proteins of pharmaceutical values; determining the effects through pharmacodynamic analysis and identifying the amino acid sequences; constructing a cDNA library from the active parts or tissues of animals and plants and obtaining the target genes that encode the active peptides; and finally obtaining the amino acid sequences of active polypeptides.

The purification and separation as disclosed supra means using the protein and polypeptide separation and purification techniques (such as affinity chromatography, hydrophobic interaction chromatography, gel electrophoresis, HPLC, mass spectrometry, etc.) to extract the proteins and polypeptides, whose active parts reflect the active principles in TCM, then analyzing the pharmacodynamical effects of the proteins and polypeptides through pharmaceutical tests to identify their pharmaceutical values, and identifying the amino acid sequences.

To construct a cDNA library is to collect the tissues of animals and plants containing active principles, extract mRNA or total RNA from cells, obtain cDNA using the RT-PCR technique, clone total cDNA fragments to an expression carrier. Theoretically, a cDNA library is a collection of the total clones of different cDNA fragments or total gene fragments in a cell. From cDNA library or from mRNA directly, the gene encoding polypeptide and protein in TCM can be obtained by using PCR techniques, and amino acid sequence can be deduced after the genes have been sequenced and verified. On one hand, polypeptide can be synthesized by chemical methods; on the other hand, by cloning the gene sequence to prokaryotic or eukaryotic expression carrier, constructing a recombinant plasmid, transforming host cells, obtaining engineered bacteria through various screening markers, the recombinant active polypeptides and proteins can be obtained by fermentation and purification of the engineered bacteria.

The genes of the active principles in TCM are the genes that encode musk's polypeptides. Musk is the dried secretion obtained from the musk gland of a musk deer (Muschus berezovskii Flerov). It is a commonly used precious traditional Chinese medicine and has 2000 years of history of use for curing diseases. Musk ranked at the top in Shen Long Ben Cao Jing, the oldest classic of Chinese medicine, and was recorded in the medical literature over the past dynasties. Musk has the functions of reviving spirit, activating blood circulation, relieving swelling and pains. It shows significant effects in quite small doses and can be used to cure certain acute conditions, such as coma, pain or inflammation. In recent years, although remarkable progress has been made in the preparation of artificial musk and its pharmaceutical effects in China, researchers have focused on synthesizing some basic chemical compositions like muscone rather than the active principles in musk, musk's polypeptides and their genes. Synthesizing musk's polypeptides by chemical techniques or gene engineering technology can overcome the limits of using fanghuosu as the substitute, preserve and exploit the gene resources of TCM.

### DESCRIPTION OF THE FIGURES

FIG. 1 Chromatography analysis of musk peptides of the invention.

FIG. 2 Polypeptide band separated from the chromatography analysis in FIG. 1.

FIG. 3 Mass spectrum of SDSECPLLCEVWILK.

FIG. 4 Mass spectrum of SDSECPLLPRQGTGSLH.

FIG. 5 Mass spectrum of IDCECPLLEAKCPSFPLWPQGREEERQ.

FIG. 6 Mass spectrum of SDSECPLLLNGTNTSSRFESINCVFLSTEEGC.

FIG. 7 Technology flowchart of obtaining amino acid sequences.

### EXAMPLES

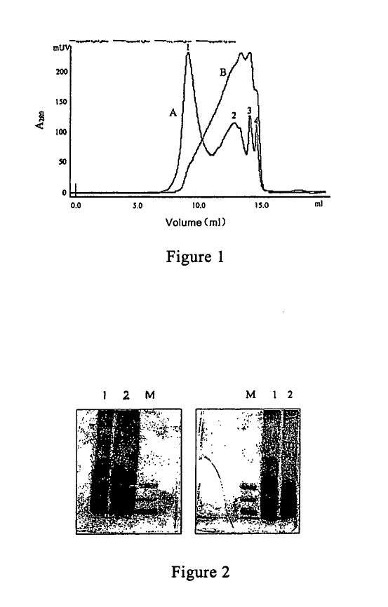

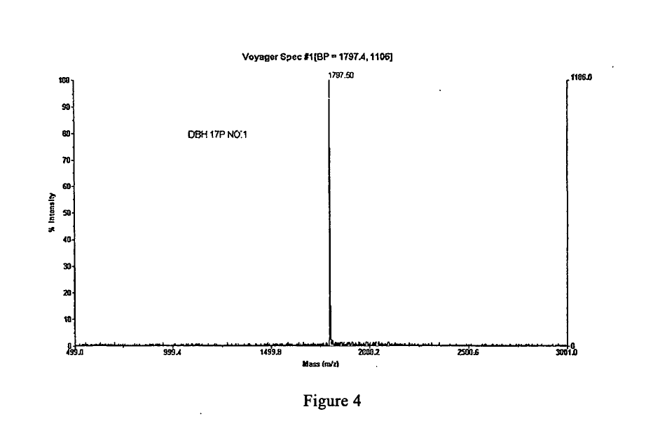

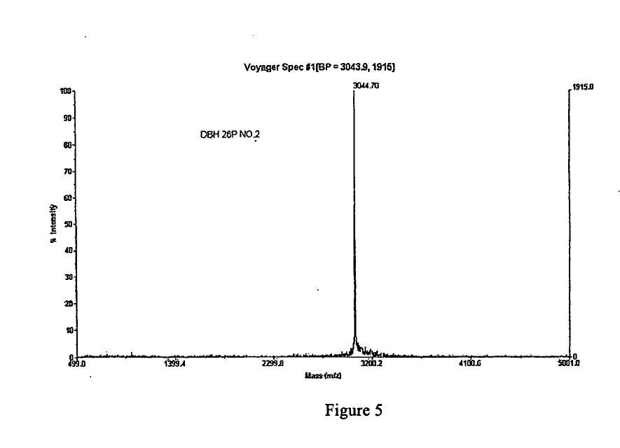

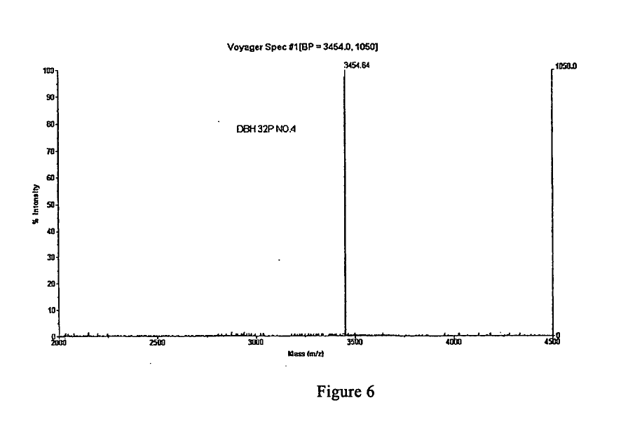

See FIGS. 1-6

Synthesis and Pharmaceutical Study of Musk's Active Polypeptides

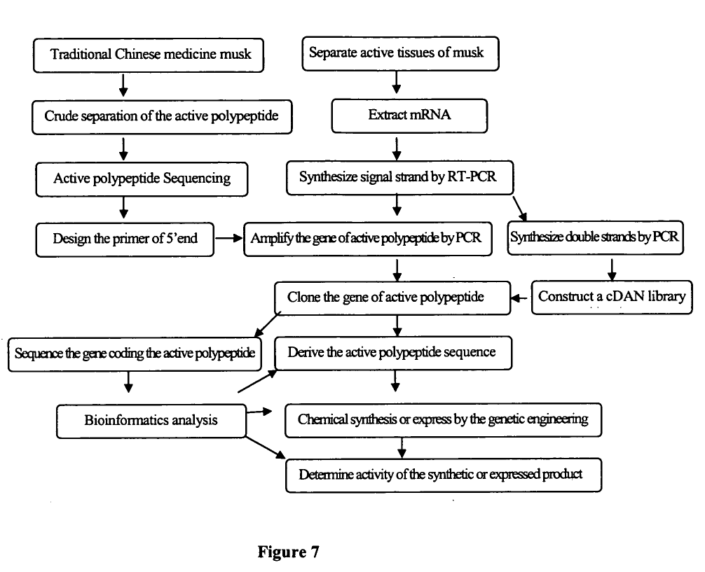

In order to first determine the target substances, the water-soluble parts of natural musk were separated and purified and their pharmaceutical effects were analyzed in the study. Details of the procedures, as presented in FIG. 7, are explained as follows.
1. Chromatography analysis (FIG. 1) and gel electrophoresis analysis (FIG. 2)

Natural musk, after extracted using organic solvents such as 95% ethanol, was dissolved in sterile water and prepared into samples for analysis. The supernatant of the sample was taken and filtered with a membrane of 0.22 μm after the sample was centrifuged. Tricine SDS-PAGE was conducted and the results were determined by means of silver staining. The results showed the basic molecular weight of proteins or peptides present in natural musk, which served as the basis for planning subsequent experiments. In this study, chromatography was conducted on the components under 8 kDa. The chromatography column used was Superdex Peptide Hr 10/30 (Pharmacia). The column was equilibrated by two bed volumes of ddH2O; 200 μL of the sample was loaded to the column; and 1.5 bed volumes of ddH2O was used to wash the column with a flow rate of 0.5 ml/min. All 4 eluted peaks were collected (FIG. 1. A: UV absorption Curve; B: Conductance Curve: 1: Elution Peak 1; 2: Elution Peak 2; 3: Elution Peak 3; 4: Elution Peak 4). Analysis primarily showed that there were 4 groups in this molecular weight range. After pharmaceutical studies were conducted for the 4 groups respectively and electrophoresis was conducted for those groups that definitely had pharmaceutical effects, single bands obtained (FIG. 2.1 and 2: Sample Analysis; M: Protein molecular weight meter) were used for amino acid sequence analysis.
2. HPLC analysis

Predicted eluted peak components can be analyzed by high-pressure liquid chromatography (HPLC) to further find out whether each of the groups is composed of a single component. If they are of multiple components, HPLC can be conducted to further separate and purify the components until single component is obtained. The single component thus obtained can be directly used for analyzing amino acid sequences. It can also be used for further mass spectrum analysis if needed.
3. Mass spectrum and amino acid sequence measurement

Mass spectrum technology can be used to examine the single component obtained so as to obtain data of the components and amino acid sequences. By means of further analysis of the amino acid sequences, the terminal or complete amino acid sequence can be determined. As a result, the gene sequences encoding the polypeptides can be deduced and used in chemical synthesis and genetic engineering production.
4. Obtaining the gene sequence

The method described supra to deduce gene sequences can lead to many possible sequences due to degenerative genetic codons. The problem can be solved through the following two ways:
a) Based on the selected expression system and the codon bias of the host cell, design and synthesize a target gene sequence, identify the sequence, and verify the synthesized sequence.
b) According to the deduced gene sequence, synthesize a primer, obtain the gene encoding the protein or polypeptide of musk deer. First, collect the musk glands of forest musk deer, then extract their mRNA and conduct RT-PCR. The cDNA obtained can be used to construct a cDNA library, or directly obtain the target gene sequence by means of PCR.
   5. Obtaining the amino acid sequences

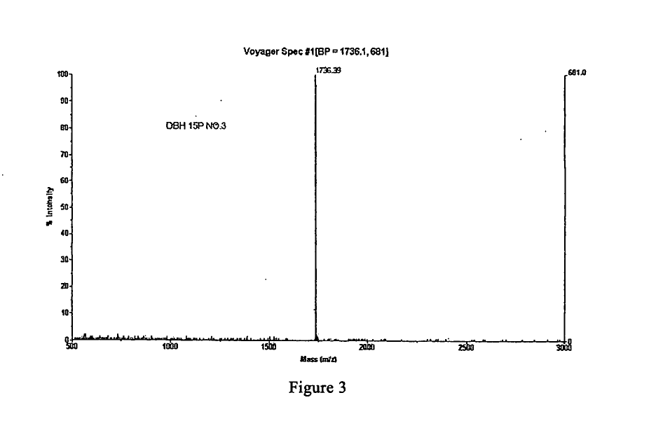

A number of amino acid sequences were obtained from the gene sequences mentioned supra. By utilizing biological information technology, candidate sequences were selected according to the pharmaceutical effects. The candidate sequences were chemically synthesized by the CL. (XIAN) BIO.SCIENTIFIC CO., LTD. The quality of the synthesis was confirmed by mass spectrometry (FIGS. 3-6) The synthetic polypeptide sequences include:

SDSECPLLCEVWILK
SDSECPLLPRQGTGSLH
IDCECPLLEAKCPSFPLWPQGREEERQ
SDSECPLLLNGTNTSSRFESINCVFLSTEEGC

Comparison of the four polypeptide sequences shows that each sequence comprises ECPLL, and polypeptide comprising more than 30% of conserved segments with the above sequences had the same properties of anti-inflammation and immune-suppressive activity. For instance, for the first sequence SDSECPLLCEVWILK and the second one SDSECPLLPRQGTGSLH, as far as sequence conservation is concerned, the former had 53% conserved, and the latter had 47% conserved. Comparison of conservation of the first sequence SDSECPLLCEVWILK and the third sequence IDCECPLLEAKCPSFPLWPQGREEERQ showed that the former had 53% conserved and that the latter had 31% conserved. Comparison of the first sequence SDSECPLLCEVWILK and the fourth one SDSECPLLLNGTNTSSRFESINCVFLSTEEGC showed that the former had 73% conserved and the latter had 34% conserved.

The polypeptides related to the above sequences can form various forms of salts, of which acetates are the most common. Data shows that acetates play certain roles in improving solubility and activity and usually contain 10-15% of acetic acid. Such polypeptide acetic salts include:

(SDSECPLLCEVWILK) Ac
(SDSECPLLPRQGTGSLH) Ac
(IDCECPLLEAKCPSFPLWPQGREEERQ) Ac
(SDSECPLLLNGTNTSSRFESINCVFLSTEEGC) Ac

6. A laboratory study of the pharmaceutical effects of polypeptide syntheses
   (1) A laboratory study on the auricular inflammation in mice—inducing auricular swelling in mice with dimethylbenzene:

The subjects were Class II K.M mice, all male, weighing 20-24 g, provided by the Animal Experiment Center of the Zhongshan University. There were a model control group, a positive (hydroxycorticoids) control group and a sample group. Suppressing function of the sample on the dimethylbenzene-induced acute inflammation was observed. The degree of ear swelling was the index. A sample of 100 μg, diluted with sterile PBS to a concentration needed, was given through IV injection. The result showed that the sample had significant suppressive effect on ear swelling in mice, which proves that the above active peptide is significantly active in anti-inflammation.
(2) Laboratory study on LPS-induced inflammation in mice

A model control group, a positive (hydroxycorticoids) control group and a sample group were formed. It was observed that the sample had suppressing effect on the activation, biosynthesis and release of inflammation-related cytokines secreted by animals that had LPS-induced early acute inflammation. Balb/c mice were provided by the Animal Experiment Center of the Zhongshan University. ELISA method was used to measure the cytokines in the mice. The dose of the sample was 25˜100 μg. The results showed that active polypeptide had significant function of suppressing cytokines (IL-6, TNF-α, IFN-γ) related to inflammation. And the suppressive effect of the sample was 500 times stronger than that of glucocorticoids in suppressing inflammatory cytokines, which proves that the medicine under discussion has the function of suppressing inflammation and autoimmune disorders.

TABLE 1
Suppressive effects of active polypeptides on the IL-6 level in serum of the
mice that served as a model of LPS-induced inflammation

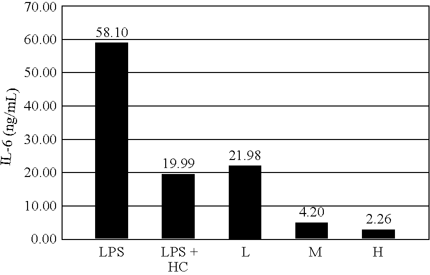

A dose of 2.5 mg/Kg of LPS (Sigma) was injected through intraperitoneal injection. At 0.5 hour after the injection, 5 groups were formed randomly: (1) negative control group (LPS), which received saline injection through intraperitoneal injection; (2) positive control group (LPS+H.C), which received 2.5 mg/kg of hydroxycorticoid through intraperitoneal injection; (3) low-dose active peptide group (L), which received a 2.5 μg/kg intraperitoneal injection; (4) medium-dose active peptide group, which received a 50 μg/kg intraperitoneal injection; and (5) high-dose active peptide group, which received a 100 μg/kg intraperitoneal injection. At 2 hours, ELISA was used to measure the IL-6 level in the serum of all groups.

TABLE 2
The suppressive effect of active peptides on the IFN-γ level in the serum
of the mice of the model group that had LPS-induced inflammation

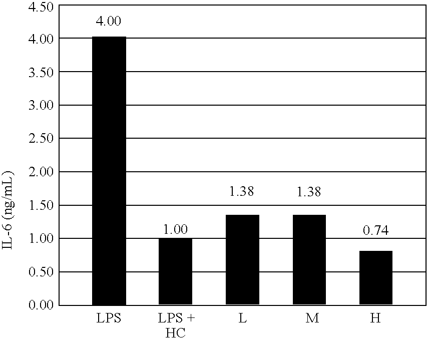

A dose of 2.5 mg/Kg of bacteriotoxin (Sigma) was injected through intraperitoneal injection. At 0.5 hour after the injection, 5 groups were formed randomly: (1) negative control group (LPS), which received saline injection through intraperitoneal injection; (2) positive control group (LPS+H.C), which received 2.5 mg/Kg of hydroxycorticoid through intraperitoneal injection; (3) low-dose active peptide group (L), which received a 2.5 μg/Kg intraperitoneal injection; (4) medium-dose active peptide group, which received a 50 μg/kg intraperitoneal injection; and (5) high-dose active peptide group, which received a 100 μg/Kg intraperitoneal injection. At 2 hours, ELISA was used to measure the IFN-γ level in the serum of all groups.
(3) A laboratory study on adjuvant arthritis in rats

This is a model of T-cell-mediated autoimmune disorders. This model has often been used in the study of pain relievers and immuno-suppressive drugs. Freund's FCA was injected percutaneously in the right side heel planta. First, local acute inflammation appeared and followed by delayed systemic metamorphic changes, resulting in poly-arthritis. Test indexes include foot swelling, auricular erythema and inflammatory nodules in the tail, and weight change. Class II male rats were used, weighing 180±20 g, provided by the First Military Medical University Animal Experimental Center (Certificate No. 0000830, 0000858), with Indommethacin as a positive control. Results showed that the sample has significant suppressive effect on arthritis with a functioning dose of 1.2˜6 μg/kg.
(4) Acute toxicity test in mice

Clean Class NIH-type mice (certificate no. 2002A022), were used, half male and female, weighing 20˜22 g, provided by the Guandong Medical Animal Experiment Center. One-off IV injection was done in the tail of the mice and followed by immediate observation of the mice for acute toxicity reaction. The test was repeated for a week. No mouse died and all mice showed no signs of toxicity. Results showed LD₅₀>5 mg/kg, indicating that the sample is safe.
(5) Neurodermatitis

Volunteers who had chronically had neurodermatitis used the sample and responded well.

1. The peptide of claim 8, the peptide comprising SDSECPLLCEVWILK, or its acetate salt (SDSECPLLCEVWLK) Ac.

2. The peptide of claim 8, the peptide comprising SDSECPLLPRQGTGSLH, or its acetate salt (SDSECPLLPRQGTGSLH) Ac.

3. The peptide of claim 8, the peptide comprising IDCECPLLEAKCPSFPLWPQGREEERQ, or its acetate salt (IDCECPLLEAKCPSFPLWPQGREEERQ) Ac.

4. The peptide of claim 8, the peptide comprising SDSECPLLLNGTNTSSRFESINCVFLSTEEGC, or its acetate salt (SDSECPLLLNGTNTSSRFESINCVFLSTEEGC) Ac.

5. A peptide or its acetate salt, the sequence of the peptide comprises ECPLL, and the sequence of the peptide is at least 30% conserved with the peptide of claim 1.

6. A method comprising applying a pharmaceutical composition, wherein the pharmaceutical composition comprises the peptide of claim 1, and wherein the pharmaceutical composition is used as an anti-inflammatory drug or immunological inhibitor.

7. A method of preparing the peptide of claim 1, the method comprising: obtaining active peptides or proteins of pharmaceutical value by separating and purifying proteins or polypeptides from musk; determining their pharmaceutical effects by means of pharmacodynamical analysis; identifying the amino acid sequences; then, constructing a cDNA library using active components or tissues from animals or plants to obtain target genes encoding the peptides; obtaining the amino acid sequences of the peptides.

8. A peptide isolated from the active principles of natural musk, or salts thereof, the sequence of the peptide comprises ECPLL.

9. A peptide or its acetate salt, the sequence of the peptide comprises ECPLL, and the sequence of the peptide is at least 30% conserved with the peptide of claim 2.

10. A peptide or its acetate salt, the sequence of the peptide comprises ECPLL, and the sequence of the peptide is at least 30% conserved with the peptide of claim 3.

11. A peptide or its acetate salt, the sequence of the peptide comprises ECPLL, and the sequence of the peptide is at least 30% conserved with the peptide of claim 4.

12. A method comprising applying a pharmaceutical composition, wherein the pharmaceutical composition comprises the peptide of claim 2, and wherein the pharmaceutical composition is used as an anti-inflammatory drug or immunological inhibitor.

13. A method comprising applying a pharmaceutical composition, wherein the pharmaceutical composition comprises the peptide of claim 3, and wherein the pharmaceutical composition is used as an anti-inflammatory drug or immunological inhibitor.

14. A method comprising applying a pharmaceutical composition, wherein the pharmaceutical composition comprises the peptide of claim 4, and wherein the pharmaceutical composition is used as an anti-inflammatory drug or immunological inhibitor.

15. A method of preparing the peptide of claim 2, the method comprising: obtaining active peptides or proteins of pharmaceutical value by separating and purifying proteins or polypeptides from musk; determining their pharmaceutical effects by means of pharmacodynamical analysis; identifying the amino acid sequences; then, constructing a cDNA library using active components or tissues from animals or plants to obtain target genes encoding the peptides; obtaining the amino acid sequences of the peptides.

16. A method of preparing the peptide of claim 3, the method comprising: obtaining active peptides or proteins of pharmaceutical value by separating and purifying proteins or polypeptides from musk; determining their pharmaceutical effects by means of pharmacodynarnical analysis; identifying the amino acid sequences; then, constructing a cDNA library using active components or tissues from animals or plants to obtain target genes encoding the peptides; obtaining the amino acid sequences of the peptides.

17. A method of preparing the peptide of claim 4, the method comprising: obtaining active peptides or proteins of pharmaceutical value by separating and purifying proteins or polypeptides from musk; determining their pharmaceutical effects by means of pharmacodynamical analysis; identifying the amino acid sequences; then, constructing a cDNA library using active components or tissues from animals or plants to obtain target genes encoding the peptides; obtaining the amino acid sequences of the peptides.
